(19) United States
(12) Patent Application Publication
(10) Pub. No.: US 2014/0205965 A1
(43) Pub. Date: Jul. 24, 2014

(54) Dual Wavelength Endodontic Treatment

(71) Applicant: BIOLASE, Inc., Irvine, CA (US)

(72) Inventors: Dmitri Boutoussov, Dana Point, CA (US); Alina Sivriver, Lake Forest, CA (US); Vladimir Netchitailo, Livermore, CA (US)

(73) Assignee: BIOLASE, Inc., Irvine, CA (US)

(21) Appl. No.: 14/160,996

(22) Filed: Jan. 22, 2014

Related U.S. Application Data
(60) Provisional application No. 61/755,174, filed on Jan. 22, 2013.

(51) Int. Cl. A61N 5/06; A61C 1/00; A61C 5/02

(52) U.S. Cl. CPC A61N 5/062; A61N 5/0603; A61C 5/02; A61C 1/0046
USPC 433/29; 433/215; 433/224; 433/216

(57) ABSTRACT

Systems and methods are provided for performing a disinfecting treatment. A fluid is placed within a volume, where the fluid absorbs radiation of a first wavelength, and where the fluid is transparent to radiation of a second wavelength. Radiation of the first wavelength is applied near or inside the volume to generate shockwaves or pressure waves in the fluid. Radiation of the second wavelength is applied near or inside the volume to cause thermal disinfection.

### CROSS-REFERENCE TO RELATED APPLICATIONS

This application claims priority to U.S. Provisional Patent Application No. 61/755,174, filed Jan. 22, 2013, entitled “Dual Wavelength Endodontic Treatment,” the entirety of which is herein incorporated by reference.

### TECHNICAL FIELD

The technology described herein relates generally to electromagnetic radiation emitting devices and more particularly to the use of electromagnetic radiation emitting devices for endodontic treatment.

### BACKGROUND

A primary cause of infection, disease, and death in humans is inadequate bacteria control. Thus, killing or removing bacteria from various systems of the human body is an important part of many medical and dental procedures. For example, during a root canal procedure, the root canal is cleaned by mechanical debridement of the canal wall and an application of an antiseptic substance within the canal to kill some of the remaining bacteria. However, dental technology has found it difficult to completely eradicate all bacteria during a root canal procedure. In particular, the structural anatomy of the tooth makes it difficult to eliminate all bacteria because the root canal includes irregular lateral canals and microscopic tubules where bacteria can lodge and fester. Bacteria control in other medical and dental procedures has proven equally difficult, and the failure to control bacteria during these procedures can lead to a variety of health and medical problems (e.g., presence of bacteria in the bloodstream, infection of organs including the heart, lung, kidneys, and spleen).

### SUMMARY

Systems and methods are provided for endodontic treatment. In a method for endodontic treatment, a fluid is placed within a root canal. The fluid absorbs radiation of a first wavelength between 1,500 nm and 3,000 nm, and is transparent to radiation of a second wavelength between 700 nm and 1,500 nm. Radiation of the first wavelength is applied inside a pulp chamber, just above the root canal, or at a depth inside the fluid-filled canal. The radiation of the first wavelength is applied in short pulses having a pulse width within a range of 1 ns to 1 ms. The pulse energy for the pulses is within a range of 1 mJ to 600 mJ. The application and absorption of the radiation of the first wavelength causes pressure waves to be generated in the fluid. These pressure waves may be generated at a single frequency or mixed frequency ranging from the audible range, 20 Hz, to ultrasound and up to 20 MHz. The pressure waves may also include a shockwave, which is defined as a pressure wave traveling at or faster than the speed of sound in the fluid medium that it is traveling through. The pressure waves damage bacteria cells, by damaging the cell membrane, and facilitate the removal of soft tissue and smear layer. The effects on the cellular membrane may be caused by shear forces, currents, and/or bubbles created by the pressure waves and effects may include, but are not limited to, deformation of the cell and cell membrane and the creation temporary pores in bacteria cell membranes. Bacteria damaged by the effects of the first wavelength are more susceptible to microbial reduction methods, including chemical and thermal methods. Radiation of the second wavelength is applied inside the pulp chamber, just above the root canal, or at a depth inside the fluid-filled canal. The radiation of the second wavelength is applied in long pulses having a pulse width in a range of 1 μs to 1 s. The long pulses have an average power within a range of 1 mW to 10 W. The radiation of the second wavelength causes thermal disinfection. The use of the radiation of the first wavelength and the radiation of the second wavelength enables a synergistic effect, where the pressure waves resulting from the radiation of the first wavelength increase efficacy of the thermal disinfection resulting from the radiation of the second wavelength.

Systems and methods are provided for performing a disinfecting treatment. A fluid is placed within a volume, where the fluid absorbs radiation of a first wavelength, and where the fluid is transparent to radiation of a second wavelength. Radiation of the first wavelength is applied near or inside the volume to generate shockwaves or pressure waves in the fluid. Radiation of the second wavelength is applied near or inside the volume to cause thermal disinfection.

As another example, a system for performing a disinfecting treatment includes a fluid for placement in a volume, the fluid absorbing radiation of a first wavelength, and the fluid being transparent to radiation of a second wavelength. A radiation module is configured to apply radiation of the first wavelength near or inside the volume to generate shockwaves or pressure waves in the fluid, and the radiation module is further configured to apply radiation of the second wavelength near or inside the volume to cause thermal disinfection.

### BRIEF DESCRIPTION OF THE FIGURES

FIG. 1 is a flowchart illustrating an example method for dual wavelength endodontic treatment.

FIG. 2 is a flowchart illustrating aspects of the example method for dual wavelength endodontic treatment.

FIG. 3 is a block diagram depicting a system for performing a disinfecting treatment.

### DETAILED DESCRIPTION

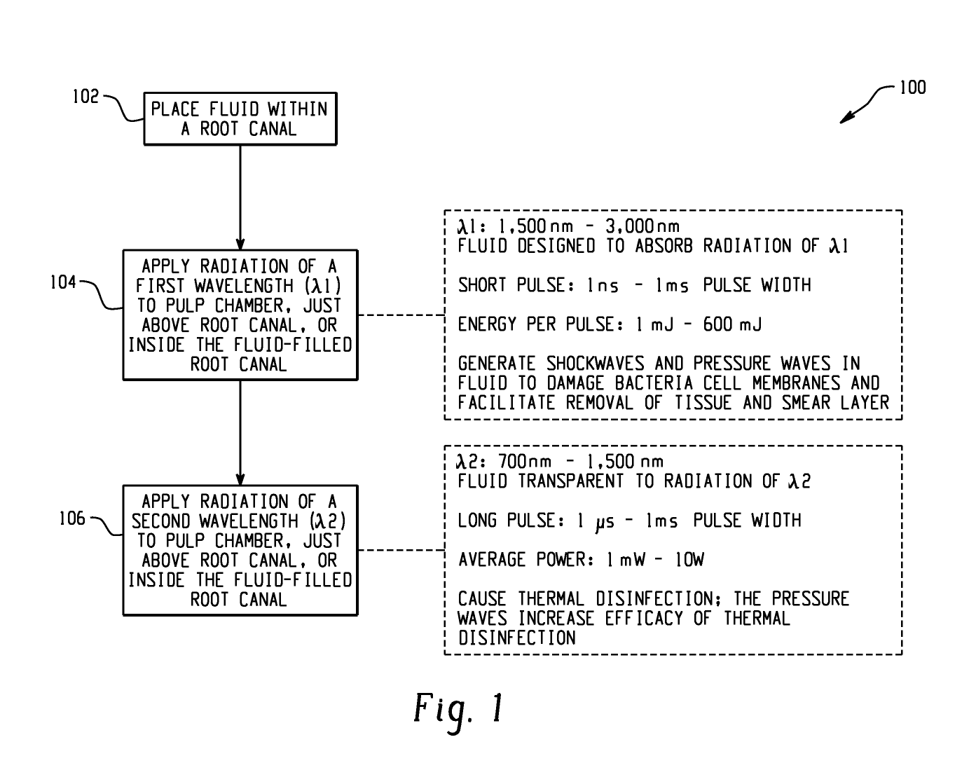

FIG. 1 is a flowchart 100 illustrating an example method for dual wavelength endodontic treatment. In implementing the dual wavelength treatment, the example method described in the flowchart 100 uses a combination of at least two laser wavelengths and may be implemented with a variety of different laser modules. The example method is used (1) to remove diseased soft tissue from a root canal and (2) to reduce a count of viable bacteria within the root canal or within a limited depth of dentinal tubules. As described in further detail below, the use of two lasers outputting radiation at two different wavelengths is used to achieve a synergistic effect. For example, radiation of a first wavelength is used to generate pressure waves within a fluid inside the root canal, and the pressure waves increase efficacy of a thermal disinfection that results from the application of radiation of a second wavelength. Thus, the radiation of the first wavelength prepares the area for the disinfection that occurs through using the radiation of the second wavelength.

In FIG. 1, at 102, a fluid is placed within the root canal. The dual wavelength treatment system involves the application of radiation of two different wavelength ranges: a mid-infrared (mid-IR) wavelength range (i.e., 1,500 nm to 3,000 nm) and a near-infrared (near-IR) wavelength range (i.e., 700 nm to 1,500 nm). The fluid placed within the root canal is designed to absorb radiation having a wavelength within the mid-IR range and to be transparent to radiation having a wavelength within the near-IR range. The dual absorption and transparency properties of the fluid may be used to create the aforementioned synergistic effect in the treatment system, where, for example, the application of the radiation of the first wavelength prepares bacteria to be killed via the application of the radiation of the second wavelength. The fluid may be a chemical antimicrobial agent used to provide a bactericidal effect within the canal. Other types of fluid may also be used (e.g., a fluid containing medication, water, a saline solution, a chemical disinfectant, etc.). The fluid may be a still fluid (i.e., flat fluid containing no bubbles) or a fluid containing bubbles.

At 104, radiation of a first wavelength is applied inside the pulp chamber, just above the root canal, or at any depth inside the fluid-filled root canal. The first wavelength is within a range of 1,500 nm to 3,000 nm (i.e., mid-IR wavelengths), and the radiation within this wavelength range may be generated by lasers including, but not limited to, Ho:YAG, Er:YSGG, Er,Cr:YSGG, Er:Glass, CTE:YAG, YAIO₃:Er, and Er:YAG lasers. The radiation of the first wavelength is applied in short pulses having a pulse width within a range of 1 ns to 1 ms. The energy per pulse is within a range of 1 mJ to 600 mJ. The short pulses of the radiation of the first wavelength may be generated via a Q-switched laser or a free-running laser (e.g., short pulse with sub-microsecond spikes within laser energy pulse, 1 mJ-100 mJ energy per spike).

The fluid of the root canal is designed to absorb the radiation of the first wavelength. The absorption of the radiation having the mid-IR wavelength in the fluid is used to create (1) pressure waves within the fluid. For the pressure waves, the radiation absorbed by the fluid may cause vapor bubble formation at a focal point of the laser energy, followed by bubble collapse. This sequence of vapor bubble formation and bubble collapse produces the pressure waves within the fluid and fluid flow out of the root canal, thereby breaking up and displacing diseased soft tissue and smear layer of the pulp cavity.

The breaking up and displacement of the diseased soft tissue and the smear layer may also be due to circulation of fluid within the canal. The radiation absorbed by the fluid may cause a secondary bubble to form within the canal, away from the focal point of the laser energy. Oscillations of the bubble surface and changes in bubble shape generate circulation of fluid surrounding the bubble. The circulation of fluid may be used to break up and displace the diseased soft tissue and the smear layer.

As noted above, the absorption of the radiation having the mid-IR wavelength in the fluid is also used to create pressure waves in the fluid. The multiple pressure waves disrupt bacteria from biofilm and launch the bacteria into a planktonic (i.e., free-floating) stage in the fluid. Planktonic bacteria are more susceptible to chemical and thermal antimicrobial agents than bacteria in biofilm. The pressure waves may also distort the membranes of bacteria cells, which increase the bacteria cells' sensitivity to antimicrobial agents. Further, the pressure waves alone may also reduce a vitality of a percentage of the bacteria cells in the fluid suspension, perhaps due to DNA damage or due to changes in the membranes of the bacteria cells. The mid-IR wavelength of the radiation is a moderately effective bactericidal agent.

At 106, radiation of a second wavelength is applied inside the pulp chamber, just above the root canal, or at any depth inside the fluid-filled root canal. The second wavelength is within a range of 700 nm to 1,500 nm (i.e., near-IR wavelengths), and the radiation within this wavelength range may be generated by lasers including, but not limited to, GaAlAs, InGaAs, and Nd:YAG lasers. The radiation of the second wavelength is applied in long pulses having a pulse width within a range of 1 μs to 1 s. The average power of the pulses is within a range of 1 mW to 10 W. The long pulses of the radiation of the second wavelength may be generated via a laser in either continuous wave (CW) or long pulse mode.

The fluid of the root canal is designed to be transparent to the radiation of the second wavelength. The radiation of the second wavelength is used to heat the root canal and thereby reduce a number of viable bacteria inside the root canal and inside a limited depth of the dentinal tubules (i.e., cause thermal disinfection). The use of the two radiation sources having the two different wavelengths may achieve the aforementioned synergistic effects, where the radiation of the first wavelength prepares the bacteria to be killed by the thermal disinfection caused by the radiation of the second wavelength. For example, the radiation of the first wavelength is absorbed in the fluid and creates the pressure waves to release the bacteria into the fluid, such that the radiation of the second wavelength can be used to heat the canal and kill the bacteria released into the fluid via the thermal disinfection. This is because free-floating bacteria are more susceptible to thermal antimicrobial agents than bacteria in biofilm. Other synergistic effects may occur through the use of the radiation of the first wavelength and the radiation of the second wavelength. For example, as noted above, the radiation of the first wavelength may be used to create pressure waves, including possible shockwaves, which cause bacterial cell membrane damage, including possible temporary pores to open in bacteria cell membranes. The damage to the bacteria cell membranes may exacerbate the sensitivity of bacteria to thermal damage from radiation of the second wavelength. Heat from the radiation of the second wavelength further damages bacteria cells by causing membrane blebbing.

The steps 104 and 106 of FIG. 1 may be performed in any desired order. Further, the steps 104 and 106 may be performed concurrently, sequentially in any order, or in an overlapping manner. The example method of FIG. 1 may utilize laser and instrumentation technology configured to deliver radiation of multiple wavelengths through a single instrument (e.g., a handpiece of a dental treatment device and/or a tip of such a dental treatment device). An example device used to implement the example method of FIG. 1 may be capable of (1) delivering multiple laser wavelengths through the same handpiece and the same tips without changing the handpiece or tips, and (2) delivering the multiple laser wavelengths at the same time, sequentially, or with overlapping pulses.

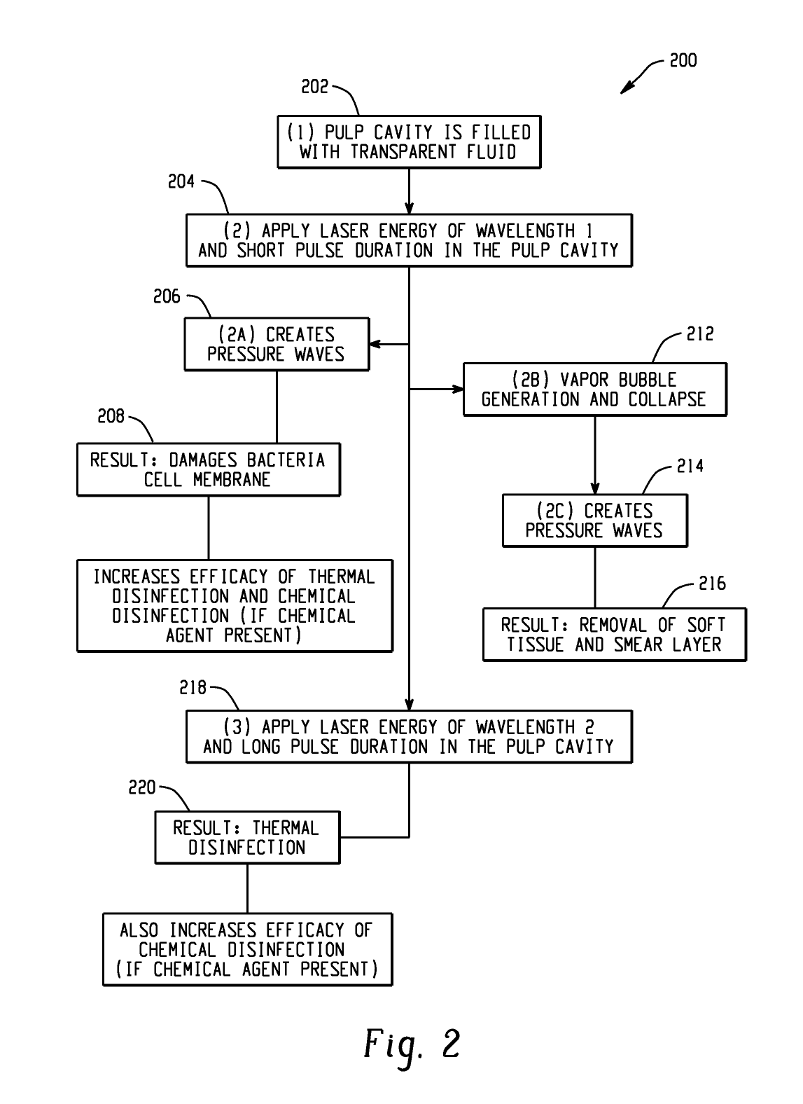

FIG. 2 is a flowchart 200 illustrating aspects of the example method for dual wavelength endodontic treatment. At 202, the pulp cavity is filled with fluid. The fluid is designed to be transparent to the wavelength range 700 nm to 1,500 nm and able to absorb energy in the wavelength range 1,500 nm to 3,000 nm. The fluid may be, for example, water, saline, or a chemical disinfectant. At 204, laser energy of a first wavelength and a short pulse duration is applied in the pulp cavity. The first wavelength is within a range of 1,500 nm to 3,000 nm, such that it is absorbed by the fluid. At 206, the application of the laser energy of the first wavelength creates pressure waves in the fluid. At 208, the pressure waves damage bacteria cells and damage the cell membrane of bacteria cells. These results of the pressure waves increase efficacy of thermal disinfection (i.e., as caused by application of energy of a second wavelength, as described below) and chemical disinfection (if a chemical agent is present). At 212, the application of the laser energy of the first wavelength also creates vapor bubble generation and collapse in the fluid. At 214, the vapor bubble generation and collapse creates pressure waves in the fluid. At 216, soft tissue and smear layer are removed due to the pressure waves.

At 218, laser energy of a second wavelength and a long pulse duration is applied in the pulp cavity. At 220, the result of the application of the second wavelength is thermal disinfection (i.e., use of heat to kill bacteria). The application of the second wavelength also increases efficacy of chemical disinfection (if a chemical agent is present).

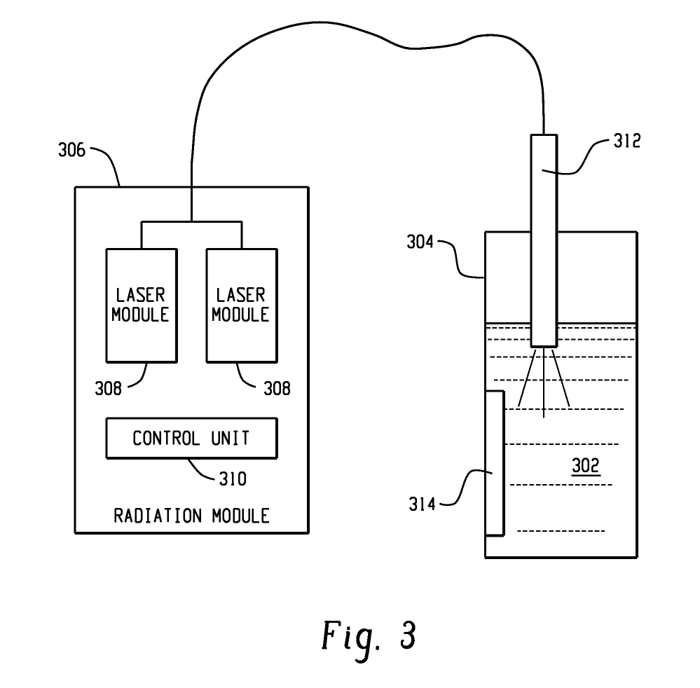

FIG. 3 is a block diagram depicting a system for performing a disinfecting treatment. The system includes a fluid 302 for placement in a volume 304, such as a root canal of a tooth. A radiation module 306 is configured to apply radiation of a first wavelength near or inside the volume 304 to generate shockwaves or pressure waves in the fluid 302. The radiation module 306 is further configured to apply radiation of a second wavelength near or inside the volume 304 to cause thermal disinfection. In one example, the radiation module includes one or more laser modules 308 controlled by a control unit 310. The one or more laser modules produce the first and second wavelengths of differing lengths to an application tip 312, as commanded by the control unit 310. In one example, a first laser module 308 generates the radiation of the first wavelength, while a second laser module 308 generates the radiation of the second wavelength. Both wavelengths of radiation may be applied using the same application tip 312 or differing application tips 312 may be used to apply the different wavelengths of radiation. In one embodiment, the radiation of the first wavelength is applied to generate shockwaves or pressure waves in the fluid 302 that can damage bacteria cells or dislodge soft tissue or a smear layer 314 from a surface, while the second wavelength of radiation is applied to provide thermal disinfection.

While the disclosure has been described in detail and with reference to specific embodiments thereof, it will be apparent to one skilled in the art that various changes and modifications can be made therein without departing from the spirit and scope of the embodiments. Thus, it is intended that the present disclosure cover the modifications and variations of this disclosure provided they come within the scope of the appended claims and their equivalents.

It should be understood that as used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Further, as used in the description herein and throughout the claims that follow, the meaning of “each” does not require “each and every” unless the context clearly dictates otherwise. Finally, as used in the description herein and throughout the claims that follow, the meanings of “and” and “or” include both the conjunctive and disjunctive and may be used interchangeably unless the context expressly dictates otherwise; the phrase “exclusive of” may be used to indicate situations where only the disjunctive meaning may apply.

1. A method for performing a disinfecting treatment, comprising:
placing a fluid within a volume, wherein the fluid absorbs radiation of a first wavelength, and wherein the fluid is transparent to radiation of a second wavelength;
applying radiation of the first wavelength near or inside the volume to generate shockwaves or pressure waves in the fluid; and
applying radiation of the second wavelength near or inside the volume to cause thermal disinfection.

2. The method of claim 1, wherein the disinfecting treatment is part of an endodontic treatment, and wherein the volume is a root canal.

3. The method of claim 2, wherein the radiation of the first wavelength is applied inside a pulp chamber, just above the root canal, or at a depth inside the fluid filled root canal.

4. The method of claim 3, wherein the pressure waves damage bacteria cells or dislodge soft tissue or a smear layer from a surface.

5. The method of claim 4, wherein the radiation of the first wavelength is applied in pulses having a pulse width within a range of 1 ns to 1,000 ns.

6. The method of claim 2, wherein the radiation of the second wavelength is applied inside a pulp chamber, just above the root canal, or at a depth inside the fluid filled root canal.

7. The method of claim 6, wherein the radiation of the second wavelength is applied in pulses having a pulse width within a range of 1 μs to 10 ms.

8. The method of claim 1, wherein the shockwaves or pressure waves increase efficacy of the thermal disinfection.

9. The method of claim 1, wherein the first wavelength is between 1,500 nm and 3,000 nm.

10. The method of claim 1, wherein the second wavelength is between 700 nm and 1,500 nm.

11. A system for performing a disinfecting treatment, comprising:
a fluid for placement in a volume, the fluid absorbing radiation of a first wavelength, and the fluid being transparent to radiation of a second wavelength;
a radiation module configured to apply radiation of the first wavelength near or inside the volume to generate shockwaves or pressure waves in the fluid;
the radiation module being further configured to apply radiation of the second wavelength near or inside the volume to cause thermal disinfection.

12. The system of claim 11, wherein the radiation module includes a laser module and an application tip.

13. The system of claim 12, wherein the application tip is configured to apply both the radiation of the first wavelength and the radiation of the second wavelength.

14. The system of claim 11, wherein the disinfecting treatment is part of an endodontic treatment, and wherein the volume is a root canal.

15. The system of claim 14, wherein the pressure waves damage bacteria cells and dislodge soft tissue or a smear layer from a surface.

16. The system of claim 15, wherein the radiation module is configured to apply the radiation of the first wavelength in pulses having a pulse width within a range of 1 ns to 1,000 ns.

17. The system of claim 11, wherein the radiation module is configured to apply the radiation of the second wavelength in pulses having a pulse width within a range of 1 μs to 10 ms.

18. The system of claim 11, wherein the shockwaves or pressure waves increase efficacy of the thermal disinfection.

19. The system of claim 11, wherein the first wavelength is between 1,500 nm and 3,000 nm.

20. The system of claim 11, wherein the second wavelength is between 700 nm and 1,500 nm.
